(19) United States
(12) Patent Application Publication
(10) Pub. No.: US 2004/0023396 A1
(43) Pub. Date: Feb. 5, 2004

(54) Ring or disk resonator photonic biosensor and its use

(76) Inventors: Robert W. Boyd, Rochester, NY (US); John E. Heebner, Rochester, NY (US)

Correspondence Address: Michael L. Goldman, NIXON PEABODY LLP, Clinton Square, P.O. Box 31051, Rochester, NY, 14603-1051, US

(21) Appl. No.: 10/294,824

(22) Filed: Nov. 14, 2002

Related U.S. Application Data
(60) Provisional application No. 60/332,822, filed on Nov. 14, 2001.

(51) Int. Cl. C12N 1/00

(52) U.S. Cl. 435/872

(57) ABSTRACT

A biosensor system includes a first optical waveguide optically coupled to a light source; a first ring or disk resonator optically coupled to the first optical waveguide; and a monitoring system that signals the presence of a biological material based on a detected change in one or more transfer characteristics of the first ring or disk resonator.

This application claims the benefit of U.S. patent application Ser. No. 60/332,822 filed Nov. 14, 2001, which is hereby incorporated by reference in its entirety.

The present invention was made, at least in part, with the use of funding receiving from Defense Advanced Research Projects Agency under Grant No. MDA972-00-1-0021. The U.S. government may have certain rights in this invention.

### FIELD OF THE INVENTION

The present invention relates to a ring or disk resonator photonic biosensor and its use in detecting the presence of biological targets in a sample.

### BACKGROUND OF THE INVENTION

There is great need for highly sensitive techniques for the detection of trace amounts of biological pathogens (Christopher et al., “Biological warfare, a historical perspective,” J. Am. Med. Assoc. 278, 412-417 (1997)). In the laboratory, standard spectroscopic techniques (Hall & Pollard, “Near-infrared spectrophotometry: a new dimension in clinical chemistry,” Clin. Chem. 38, 1623-1631 (1992)) can be used to detect various biological materials with great sensitivity and specificity, but for use outside of a laboratory environment there is great need for robust, integrated devices that are both inexpensive and can be used in harsh environments. Integrated optics or photonics allow optobiological interactions in a compact geometry. Some techniques that are under intense investigation include biosensors constructed from directional couplers (Luff et al., “Integrated-optical directional coupler biosensor,” Opt. Lett. 21, 618-620 (1996)), Mach-Zehnder interferometers (Luff et al. “Integrated optical Mach-Zehnder biosensor,” J. Lightwave Technol. 16, 583-592 (1998)), and various configurations that make use of surface-plasmon resonance (Lukosz, “Principles and sensitivities of integrated optical and surface plasmon sensors for direct affinity sensing and immunosensing,” Biosens. Bioelectron. 6, 215-225 (1991); Kolomenskii et al., “Sensitivity and detection limit of concentration and adsorption measurements by laser-induced surface-plasmon resonance,” Appl. Opt. 36, 6539-6547 (1997); Kolomenskii et al., “Surface-plasmon resonance spectrometry and characterization of absorbing liquids,” Appl. Opt. 39, 3314-3320 (2000)).

Microresonators have previously found application in disk lasers (McCall et al., “Whispering-gallery mode microdisk lasers,” Appl. Phys. Lett. 60, 289-291 (1992); Yamamoto & Slusher, “Optical processes in microcavities,” Phys. Today 46, 66-74 (1993); Knight et al., “Core-resonance capillary-fiber whispering-gallery-mode laser,” Opt. Lett. 17, 1280-1282 (1992); Popp et al., “Q-switching by saturable absorption in microdroplets: elastic scattering and laser emission,” Opt. Lett. 22, 1296-1298 (1997)), high-resolution spectroscopy (Schiller & Byer, “High-resolution spectroscopy of whispering gallery modes in large dielectric spheres,” Opt. Lett. 16, 1138-1140 (1991)), laser frequency stabilization (Vassiliev et al., “Narrow-line-width diode laser with a high-Q microsphere resonator,” Opt. Commun. 158, 305-312 (1998)), add-drop filters for wavelength division multiplexing (Little et al., “Microring resonator channel dropping filters,” J. Lightwave Technol. 15, 998-1005 (1997)), dispersion compensation (Madsen & Lenz, “Optical all-pass filters for phase response design with applications for dispersion compensation,” IEEE Photon. Technol. Lett. 10, 994-996 (1998)), all-optical switching (Braginsky et al., “Quality-factor and nonlinear properties of optical whispering-gallery modes,” Phys. Lett. A 137, 393-397 (1989); Blom et al., “Experimental study of integrated-optics microcavity resonators: toward an all-optical switching device,” Appl. Phys. Lett. 71, 747-749 (1997)), and cavity quantum electrodynamics (Benner et al., “Observation of structure resonances in the fluorescence spectra from microspheres,” Phys. Rev. Lett. 44, 475-478 (1980); Campillo et al., “Cavity quantum electrodynamic enhancement of stimulated emission in microdroplets,” Phys. Rev. Lett. 67, 437-440 (1991); Vernooy et al., “High-Q measurements of fused-silica. microspheres in the near infrared,” Opt. Lett. 23, 247-249 (1998)). Moreover, Blair and Chen have recently proposed use of cylindrical optical cavities for resonantly enhanced fluorescence biosensing (“Resonant-enhanced evanescent-wave fluorescence biosensing with cylindrical optical cavities,” Appl. Opt. 40, 570-582 (2001)). None of these procedures relies on absorption for performing high-sensitivity detection of biological materials and, more particularly, the monitoring of transfer characteristics of a microresonator to detect trace amounts of biological material on the surface of the microresonator.

The present invention is directed to overcoming these and other deficiencies in the art for purposes of obtaining a photonic detection system for readily identifying minute quantities of biological targets.

### SUMMARY OF THE INVENTION

A first aspect of the present invention relates to a biosensor system for monitoring for a presence of a biological target. The biosensor system includes: an optical waveguide optically coupled to a light source; a ring or disk resonator optically coupled to the optical waveguide; and a monitoring system that signals the presence of a biological target based on a detected change in one or more transfer characteristics of the ring or disk resonator.

A second aspect of the present invention relates to a method of detecting the presence of a biological target in a sample. The method includes: exposing a biosensor system of the present invention to a sample and monitoring the ring or disk resonator for a change in one or more transfer characteristics, wherein a change in the one or more transfer characteristics indicates adsorption of a biological target to a surface of the ring or disk resonator and presence of the biological target in the sample.

A third aspect of the present invention relates to a method of making the biosensor system of the present invention. The method includes: optically coupling a ring or disk resonator to one or more optical waveguides that are coupled to a light source and a monitoring system, and attaching one or more probes to a surface of the ring or disk resonator.

The photonic biosensor system of the present invention is based on a high-finesse, whispering-gallery-mode ring or disk resonator that can be used for the detection of biological targets, including pathogens. High sensitivity is achieved because the light wave interacts many times with each biological target as a consequence of the resonant recirculation of light within the ring or disk structure. Specificity of the detected biological target can be achieved when a layer of one or more probes is deposited onto an active area of the resonator. Formulas are presented that allow the sensitivity of the device to be quantified; these formulas show that, under optimum conditions, as few as 100 molecules can be detected.

### BRIEF DESCRIPTION OF THE DRAWINGS

FIGS. 1A-C illustrate alternative embodiments of a single ring or disk resonator biosensor system of the present invention. FIG. 1A illustrates a biosensor that contains a single optical waveguide to deliver light to and receive light from the disk resonator. Light from an optical waveguide is weakly coupled (with coupling coefficients r and t) to a whispering-gallery mode of a high-finesse disk resonator. The presence of biological materials near the surface of the disk leads to a dramatic change in the transfer characteristics of the device. This embodiment can be used to monitor the presence of biological materials through an induced change in the internal losses of the disk. FIG. 1B illustrates a biosensor that also includes a second optical waveguide to receive light from the disk resonator. As with the first embodiment, light from the optical waveguide is weakly coupled (with coupling coefficients r and t) to the whispering-gallery mode of a high-finesse disk resonator. The embodiment illustrated in FIG. 1B can be used to monitor the presence of biological materials through an induced change in the effective refractive index of the disk material. FIG. 1C illustrates a third embodiment of a biosensor which is an extension of that shown in FIG. 1A. The single guide-coupled disk is inserted into one arm of a Mach-Zehnder interferometer so that induced changes in phase may be converted to changes in transmission. Just as in the case of FIG. 1B, this embodiment can also be used to monitor the presence of biological materials through induced changes in the effective refractive index of the disk.

FIG. 2 is a graph illustrating a buildup factor B for the system shown in FIG. 1A plotted against the single-pass absorption A=−ln τ for several values of the coupling coefficient R=r².

FIG. 3 is a graph of resonator transmission T=|E₃/E₁|² for the system shown in FIG. 1A plotted against the single-pass absorption A=−ln τ for several values of the coupling coefficient R=r².

FIG. 4 is a graph of resonator transmission T=|E₃/E₁|² plotted against the single-pass phase shift Δφ for several values of the coupling coefficient R=r², for the balanced, four-port device shown FIG. 1B.

FIGS. 5A-B are diagrams illustrating a simulation of a field distribution in the region of the disk and waveguide in the absence of an absorbing particle (5A) and in the presence of an absorbing particle (5B).

FIGS. 6A-B illustrate silanization (6A) and halide (6B) coupling agents which can be attached to, e.g., a silica coated resonator surface and used to covalently bind probes. For purposes of illustration, trimethoxy(3-oxiranylpropyl)silane is illustrated; other silanes have been used in practice.

FIGS. 7A-E illustrate the attachment schemes for binding probes R—NH2, R—SH, and R—OH upon opening of the epoxide group on the coupling agent (7A-C, respectively); probe R-alkenyl to the alkenyl group on the coupling agent (7D); and probe R—OH upon displacement of a halide coupling agent (7E). For purposes of illustration, trimethoxy(3-oxiranylpropyl)silane is illustrated in FIGS. 7A-C; other silanes have been used in practice.

### DETAILED DESCRIPTION OF THE INVENTION

The photonic biosensor system of the present invention is based on a high-finesse, whispering-gallery-mode ring or disk resonator that can be used for the detection of biological targets.

The biosensor system includes one or more optical waveguides, at least one of which is optically coupled to a light source, one or more ring or disk resonators that are optically coupled to (i.e., positioned adjacent to a portion of) the one or more waveguides, and a monitoring system that signals the presence of a biological target based on a detected change in one or more transfer characteristics of the one or more ring or disk resonators.

The one or more ring or disk resonators can be constructed by known fabrication techniques (McCall et al., “Whispering-gallery mode microdisk lasers,” Appl. Phys. Lett. 60, 289-291 (1992); Yamamoto & Slusher, “Optical processes in microcavities,” Phys. Today 46, 66-74 (1993); Rayleigh, “The problem of the whispering gallery,” Philos. Mag. 20, 1001-1004 (1910); Braginsky & Ilchenko, “Properties of optical dielectric microresonators,” Sov. Phys. Dokl. 32, 306-307 (1987); Rafizadeh et al., “Waveguide-coupled Al—GaAs/GaAs microcavity ring and disk resonators with high finesse and 21.6-nm free spectral range,” Opt. Lett. 22, 1244-1246 (1997), each of which is hereby incorporated by reference in its entirety).

Basically, this process involves deposition of the vertical light-guiding structure onto a substrate and subsequent etching of the structure to form the lateral light-guiding structure which includes the waveguide(s) and resonator(s). The deposition may be carried out via sputtering, chemical vapor deposition, or molecular beam epitiaxy (MBE). For the deposition of crystalline AlGaAs, MBE is a preferred method of deposition. To define the vertical photonic structure that will guide light in this system, three layers of AlGaAs are deposited above a GaAs substrate. The middle, high-index light-guiding layer is of the order of 1 micron thick. The subsequent etching is accomplished in two etch steps. First, a thin layer of SiO₂ is deposited above the MBE grown structure via chemical vapor deposition. An electron-beam resist such as PMMA is then spin coated on top to a thickness of about 150 nm. A pattern of trenches which laterally define the structures is used to raster-expose the resist. The resist is then developed and used as an etch mask for transferring the pattern into the underlying SiO₂ layer. This is accomplished via reactive ion etching (RIA) with CHF₃. Finally, the pattern is transferred into the AlGaAs structure using chemically assisted ion beam etching (CAIBE).

Ring or disk resonators can be formed of glasses or crystalline semiconductor materials. Glasses are typically easier to work with and have low losss characteristics, but formation of ring or disk resonators of less than about 10 μm is difficult at best. Semiconductor materials are capable of forming ring or disk resonators that achieve preferred dimensions (see below). Exemplary glasses include, without limitation, silica (SiO₂), arsenic selenide (As₂Se₃), and silicon nitride (Si₃N₄). Exemplary semiconductors systems include, without limitation, aluminum gallium arsenide (AlGaAs) and indium phosphide (InP). Other glasses and crystalline semiconductor materials that are suitable for carrying optical signals are continually being identified and can also be used in the present invention.

Suitable dimensions for the ring or disk resonator include: a resonator radius of about 2 μm to about 50 μm, preferably about 2 μm to about 20 μm; and a resonator-air refractive index ratio of about 3.4 to about 1.5, more preferably about 3.1 to about 2.5.

A ring or disk may be implemented as the resonator, each having advantages. A disk supports higher order radial modes which may be unintentionally excited and may interfere with the detection process. A ring can substantially restrict the number of modes but the presence of another etched sidewall will introduce extra roughness-induced scattering losses. Suitable dimensions for the guide and ring widths are about 0.3 μm to about 1 μm, preferably tapered out to 8 μm for ease of coupling to fiber-based systems.

The ring or disk resonators are optically coupled to one or more waveguides, at least one of which is coupled to a light source. Suitable light sources include, without limitation, a continuous wave (cw), pulsed or non-pulsed laser, or an LED. It is also preferable that the light source emit polarized light, either directly or as a result of polarizing filters positioned between the light source and optical waveguide coupled thereto. Average power levels of 1 milliwatt are sufficient. Coupling may be achieved by fixing a fiber source or semiconductor-based laser to an appropriately tapered waveguide.

If only one optical waveguide is coupled to the one or more ring or disk resonators, then each of the ring or disk resonators will have only one port. The one or more ring or disk resonators can be coupled to a single optical waveguide either in series or in parallel (with the single optical waveguide being split from a common source). If two optical waveguides are coupled to each of the one or more ring or disk resonators, then each of the ring or disk resonators will have two ports. Where two optical waveguides are coupled to the ring or disk resonators, only one of the optical waveguides is coupled directly to the light source. The second optical waveguide can be the same or different for each of the one or more ring or disk resonators.

The optical coupling of the optical waveguide to the ring or disk resonators is preferably weak, which allows the light intensity to build up to high values within the resonator. The ratio of the circulating intensity within the resonator to the incident intensity is defined to be the buildup factor B of the configuration (the buildup factor B is related to the finesse F, often used to describe optical resonators through the relation B=(2/π)F). By weak optical coupling, it is intended that a gap is present between the optical waveguide and the port of the ring or disk resonator. Suitable gaps can range from about 0.05 μm to about 0.5 μm, preferably about 0.1 μm to about 0.3 μm. The gaps can, of course, be adjusted to accommodate different parameters resulting from changes in materials and the desired sensitivity of the biosensor system. Typically, greater sensitivity is afforded by weaker optical coupling, though an ultimate restriction is imposed by intra-resonator losses.

The ring or disk resonator can also have one or more probes coupled to a surface thereof, preferably within the region of the ring or disk where light is confined. The one or more probes each include (i) one or more surface-binding groups which enable them to be coupled to the surface of the ring or disk resonator (either directly or via a coupling agent) and (ii) one or more target-binding groups that bind to a target molecule. Although not limited to such, the one or more surface-binding groups are typically hydroxyl groups. The one or more target-binding groups can include, without limitation, an amino group, a thiol, a hydroxyl, an alkyl chain, an ester, a carboxylic acid, an aromatic, a heterocycle, or a combination thereof.

The one or more probes can be the same or different on a single ring or disk resonator, each capable of attaching to the same target or a different target. In addition, where multiple ring or disk resonators are employed, the one or more probes on each ring or disk can be the same, but different probes are used on each ring or disk resonator.

Suitable probes generally include, without limitation, non-polymeric small molecules, polypeptides or proteins, and oligonucleotides.

Exemplary non-polymeric small molecules include, without limitation: avidin, peptido-mimetic compounds, and vancomycin. One class of peptido-mimetic compounds is disclosed in U.S. patent application Ser. No. 09/568,403 to Miller et al., filed May 10, 2000, each of which is hereby incorporated herein by reference in its entirety. A preferred peptido-mimetic compound which binds to lipopolysaccharide is a tetratryptophan ter-cyclopentane as disclosed in the above-noted application to Miller et al. Other peptidomimetic compounds can also be employed.

Exemplary polypeptides include, without limitation, a receptor for cell surface molecule or fragment thereof; a lipid A receptor; an antibody or fragment thereof; peptide monobodies of the type disclosed in U.S. patent application Ser. No. 09/096,749 to Koide, filed Jun. 12, 1998, and U.S. patent application Ser. No. 10/006,760 to Koide, filed Nov. 19, 2001, each of which is hereby incorporated by reference in its entirety; a lipopolysacchardide-binding polypeptide; a peptidoglycan-binding polypeptide; a carbohydrate-binding polypeptide; a phosphate-binding polypeptide; a nucleic acid-binding polypeptide; and polypeptides which bind organic warfare agents such as tabun, sarin, soman, GF, VX, mustard agents, botulinium toxin, Staphylococcus entertoxin B, and saitotoxin.

Exemplary oligonucleotide probes can by DNA, RNA, or modified (e.g., propynylated) oligonucleotides of the type disclosed in Barnes et al., J. Am. Chem. Soc. 123:4107-4118 (2001), and Barnes et al., J. Am. Chem. Soc. 123:9186-9187 (2001), each of which is hereby incorporated by reference in its entirety. The oligonucleotide probes can be any length which is suitable to provide specificity for the intended target. Typically, oligonucleotide probes which do not contain modified nucleotides will be at least about 12 to about 100 nucleotides in length. For oligonucleotides which contain modified bases, oligonucleotides should be at least about 7 nucleotides in length, up to about 100 nucleotides in length.

Target molecules that can be bound be the one or more probes include, without limitation: proteins (including without limitation enzymes, cell surface molecules, antibodies or fragments thereof), glycoproteins, peptidoglycans, carbohydrates, lipoproteins, a lipoteichoic acid, lipid A, phosphates, nucleic acids which are expressed by certain pathogens (e.g., bacteria, viruses, multicellular fungi, yeasts, protozoans, multicellular parasites, etc.), or organic compounds such as naturally occurring toxins or organic warfare agents, etc. The target molecules can be a component of a pathogen (e.g., cell wall or viral capsid component), or liberated therefrom using known processing techniques. These target molecules can be detected from any source, including food samples, water samples, homogenized tissue from organisms, etc.

A number of strategies are available for attaching the one or more probes to the surface of the ring or disk resonator, depending upon the type of probe which is ultimately to be attached thereto. The available strategies for attaching the one or more probes include, without limitation, covalently bonding a probe to the surface of the ring or disk resonator, ionically associating the probe with the surface of the ring or disk resonator, adsorbing the probe onto the surface of the ring or disk resonator, or the like. Such association can also include covalently or noncovalently attaching the probe to another moiety (of a coupling agent), which in turn is covalently or non-covalently attached to the surface of the ring or disk resonator.

Typically, the surface to which the probe will be attached includes an oxide glass layer, which is either present due to use of an oxide glass to prepare the resonator or applied thereto (e.g., via sputtering) as a thin coating to the surface of a crystalline semiconductor material. Thereafter, the surface is functionalized (i.e., primed) with a coupling agent which is attached to the surface thereof. This is achieved by providing a coupling agent precursor and then covalently or non-covalently binding the coupling agent precursor to the surface of the resonator structure. Once the surface has been primed, the probe is exposed to the primed resonator surface under conditions effective to (i) covalently or non-covalently bind to the coupling agent or (ii) displace the coupling agent such that the probe covalently or non-covalently binds directly to the resonator surface. The binding of the probe to the resonator surface is carried out under conditions which are effective to allow the one or more target-binding groups thereon to remain available for binding to the target molecule. The use of an oxide glass layer affords standard glass-binding chemistry to be employed in coupling the one or more probes to the primed surface.

Suitable coupling agent precursors include, without limitation, silanes functionalized with an epoxide group, a thiol, or an alkenyl; and halide containing compounds.

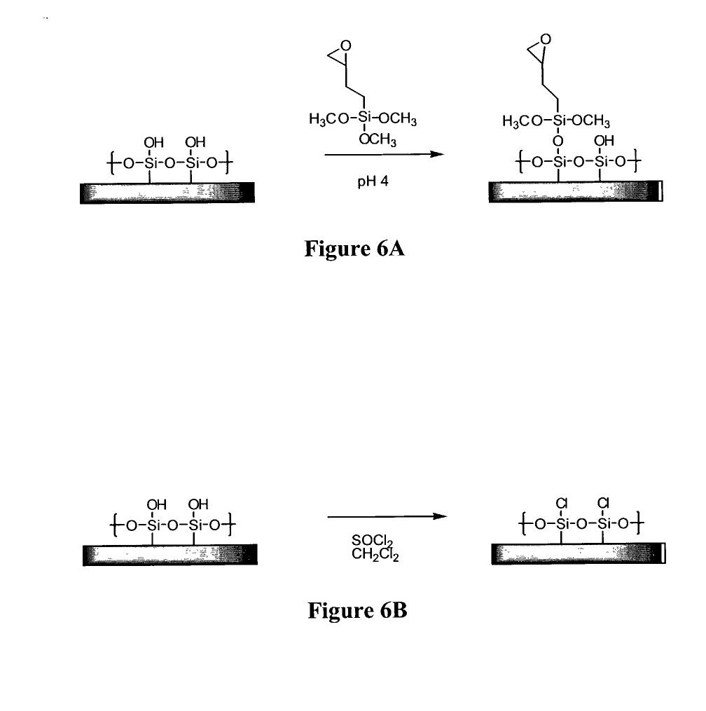

Silanes include a first moiety which binds to the surface of the resonator structure and a second moiety which binds to the probe. Preferred silanes include, without limitation, 3-glycidoxypropyltrialkoxysilanes with C1-6 alkoxy groups, trialkoxy(oxiranylalkyl)silanes with C2-12 alkyl groups and C1-6 alkoxy groups, 2-(1,2-epoxycyclohexyl)ethyltrialkoxysilane with C1-6 alkoxy groups, 3-butenyl trialkoxysilanes with C1-6 alkoxy groups, alkenyltrialkoxysilanes with C2-12 alkenyl groups and C1-6 alkoxy groups, tris[(1-methylethenyl)oxy]3-oxiranylalkyl silanes with C2-12 alkyl groups, [5-(3,3-dimethyloxiranyl)-3-methyl-2-pentenyl]trialkoxysilane with C1-6 alkoxy groups, (2,3-oxiranediyldi-2,1-ethanediyl)bis-triethoxysilane, trialkoxy[2-(3-methyloxiranyl)alkyl]silane with C1-6 alkoxy groups and C2-12 alkyl groups, trimethoxy[2-[3-(17,17,17-trifluoroheptadecyl)oxiranyl]ethyl]silane, tributoxy[3-[3-(chloromethyl)oxiranyl]-2-methylpropyl]silane, and combinations thereof. Silanes can be coupled to the resonator structure according to a silanization reaction scheme shown in FIG. 6A, the conditions for which are well known to those of skill in the art.

Halides can also be coupled to the resonator structure according to the reaction scheme set in FIG. 6B, the conditions for which are well known to those of skill in the art.

Thereafter, the one or more probes are bound to the resonator structure according to the type of functionality provided by the coupling agent. Typically, probes are attached to the coupling agent or displace to coupling agent for attachment to resonator structure in aqueous conditions or aqueous/alcohol conditions.

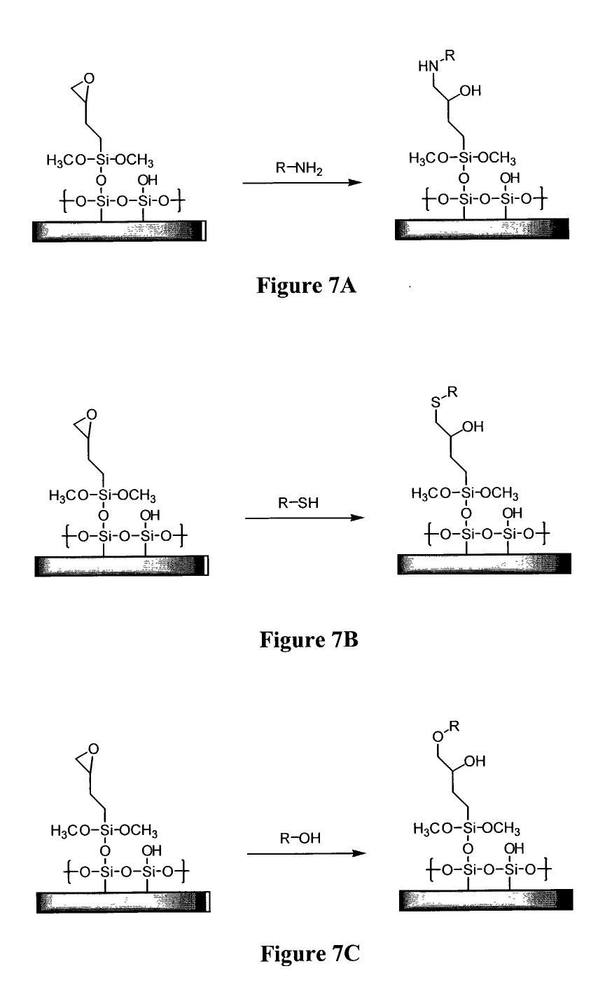

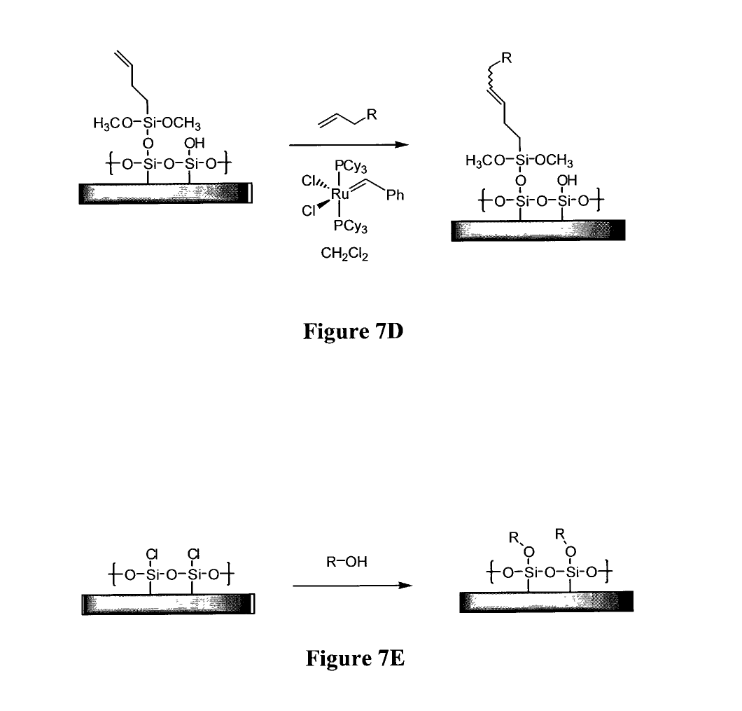

Epoxide functional groups can be opened to allow binding of amino groups according to the reaction scheme set forth in FIG. 7A, the conditions for which are well known to those of skill in the art. Epoxide functional groups can also be opened to allow binding of thiol groups or alcohols according to the reaction scheme set forth in FIGS. 7B-C, respectively, the conditions for which are well known to those of skill in the art.

Alkenyl functional groups can be reacted to allow binding of alkenyl groups according to the reaction scheme set forth in FIG. 7D, the conditions for which are well known to those of skill in the art.

Where a halide coupling agent is employed, the halide coupling agent is typically displaced upon exposing the primed resonator structure to one or more probes which contain alcohol groups as the resonator-binding groups. The displacement can be carried out according to the reaction scheme set forth in FIG. 7E, the conditions for which are well known to those of skill in the art.

The monitoring system can be any suitable detector capable of detecting the one or more transfer characteristics that change upon adsorption or binding of a target molecule to the surface of the ring or disk resonator. Transfer characteristics that can be monitored include: the intensity of light scattering out of the resonator (i.e., a change in internal losses), intensity of light leaving an output port, phase change of the circulating optical power, the effective refractive index of the resonator, or a combination thereof. Monitoring systems typically include a biasing power supply, an optical detector that can receive optical signals and convert the optical power into electrical current with varying voltages depending on the strength of the optical power, and a device that measures voltage of the resulting current. The measurement device can also include a display. Exemplary monitoring systems include, without limitation, InGaAs, Ge, or Si detectors.

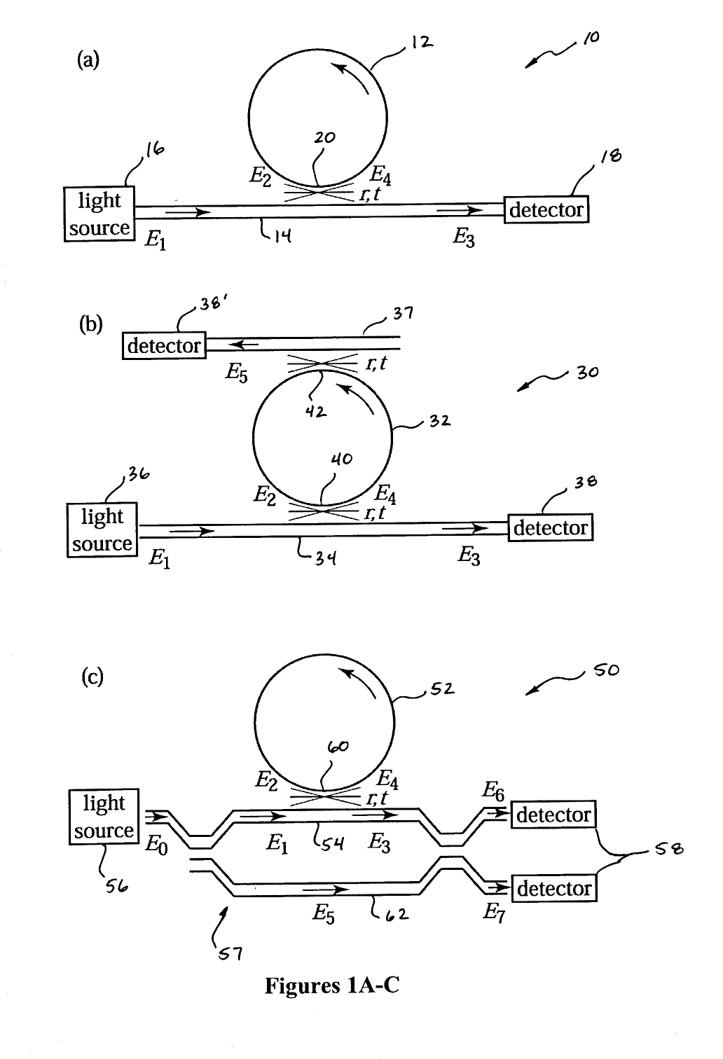

One preferred biosensor system of the present invention is illustrated in FIG. 1A. The biosensor system 10 includes a single ring or disk resonator 12, an optical waveguide 14 that is coupled to a light source 16, and a monitoring system 18 that can monitor the one or more transfer characteristic of the ring or disk resonator. The resonator 12 is situated close enough to the waveguide 14 to achieve evanescent coupling via a single port 20. The monitoring system 18 is coupled to the optical waveguide downstream of the ring or disk resonator. Changes in transmission due to induced changes in the losses (i.e., scattering) experienced by light circulating within the resonator can be monitored. Systems having this design can include multiple resonators in parallel.

Another preferred biosensor system of the present invention is illustrated in FIG. 1B. The biosensor system 30 includes a single ring or disk resonator 32, an optical waveguide 34 that is coupled to a light source 36, a second optical waveguide 37, and one or more monitoring systems 38, 38′ that can monitor the one or more transfer characteristic of the ring or disk resonator. The resonator 32 is situated close enough to the waveguides 34, 37 to achieve evanescent coupling. The optical waveguide 34 is optically coupled to the ring or disk resonator 32 via port 40 and the optical waveguide 37 is optically coupled to the ring or disk resonator 32 via port 42. The monitoring systems 38,38′ can be coupled to the optical waveguide 34 downstream of the ring or disk resonator or the optical waveguide 37 downstream of the ring or disk resonator, respectively. Changes in transmission(s) due to induced changes in the effective refractive index experienced by light circulating within the resonator can be monitored. One approach for detecting these changes is by measuring output through port 42 and waveguide 37. Alternatively, a shift in the resonance dips or peaks can be monitored. Systems having this design can readily incorporate multiple resonators in series with the first optical waveguide, particularly where each is optically coupled to separate and distinct output waveguides.

Yet another preferred biosensor system of the present invention is illustrated in FIG. 1C, exemplifying a biosensor system as shown in FIG. 1A where the optical waveguide is incorporated into a Mach-Zehnder interferometer. The biosensor system 50 includes a resonator 52, an optical waveguide 54 that is coupled to a light source 56 and acts as a component of the interferometer 57, and a monitoring system 58 that can monitor the one or more transfer characteristic of the ring or disk resonator. The resonator 52 is situated close to the waveguide to achieve evanescent coupling. The optical waveguide 54 is optically coupled to the ring or disk resonator 52 via port 60. The interferometer 57 includes the waveguide 54 and a second waveguide 62 optically coupled thereto at locations upstream and downstream of the resonator. The second waveguide 62 effectively carries a reference signal that allows changes in transfer characteristics to be detected by monitoring system 58. The monitoring system 58 is coupled to each of the optical waveguides 54, 62 downstream of the ring or disk resonator. Changes in transmission(s) due to induced changes in the effective refractive index experienced by light circulating within the resonator can be monitored. Alternatively, a shift in the resonance dips or peaks can be monitored.

A further aspect of the present invention relates to a method of detecting the presence of a biological target in a sample. Basically, a biosensor system of the present invention is exposed to a sample under conditions effective to allow binding of a target molecule in the sample to the one or more probes of the biological sensor. After such exposure, the ring or disk resonator(s) is(are) monitored for a change in one or more transfer characteristics, wherein a change in the one or more transfer characteristics indicates adsorption of a biological target to a surface of the ring or disk resonator(s) and presence of the biological target in the sample. Generally, the greater the change, the greater the number of biological targets that have been adsorbed onto the resonator.

As noted above, the biosensor system can be used to detect the presence of a pathogen in a sample. Samples which can be examined include blood, water, a suspension of solids (e.g., food particles, soil particles, etc.) in an aqueous solution, or a cell suspension from a clinical isolate (such as a tissue homogenate from a mammalian patient).

In particular, a preferred method of the present invention involves the detection of a Gram negative bacteria in a sample. This is achieved by exposing the sample to a biosensor system of the present invention which includes one or more probes that bind to lipid A or fragments thereof. A preferred probe of this type is a tetratryptophan ter-cyclopentane peptido-mimetic compound as disclosed in U.S. patent application Ser. No. 09/568,403 to Miller et al., filed May 10, 2000, which is hereby incorporated herein by reference in its entirety. Thereafter, a determination is made as to whether a change in transfer characteristics has occurred following said exposure, indicating the presence of lipid A and, thus, Gram negative bacteria in sample. To ensure that any lipid A is available to bind to the probe, it is desirable but not essential to treat the sample prior to its exposure to the biological sensor in a manner effective to disrupt the cellular membrane of Gram negative bacteria in the sample, thereby releasing lipid A contained within the bacterial membrane. This can be achieved by chemical means which do not modify the structure of lipid A itself, by mechanical means (French press), by sonication, or freezing (and thawing).

### EXAMPLES

The following examples are intended to illustrate, but by no means are intended to limit, the scope of the present invention as set forth in the appended claims.

### Example 1

### Mathematical Model and Predictions for Single Disk Resonator

The device illustrated in FIGS. 1A-B can be analyzed mathematically as follows (Heebner & Boyd, “Enhanced all-optical switching by use of a nonlinear fiber ring resonator,” Opt. Lett. 24, 847-849(1999), which is hereby incorporated by reference in its entirety). The coupling of light into and out of the resonator can be described in terms of generalized beam-splitter relations of the form

E₃=rE₁+itE₂, Eq. (1)

E₄=rE₂+itE₁, Eq. (2)

where r and t are taken to be real quantities that satisfy the relation r²+t²=1 and where the fields are defined with respect to the reference points indicated in FIGS. 1A-B. (The coupling behavior of light in the interferometer shown in FIG. 1C is well known and does not modify the relationship for the above equations.) In addition, the circulation of light within the resonator can be described in terms of the round-trip phase shift φ and the amplitude transmission factor τ such that

E₂=τ exp(iφ)E₄ Eq. (3)

The round-trip phase shift φ can be interpreted as kL_{eff}, where k=2πn/λ, n is the effective refractive index of the ring or disk structure, λ is the vacuum wave-length of the incident light, and L_{eff} is the effective circumference of the mode of the disk resonator, which is roughly equal to the physical circumference of the disk. The disk is assumed to be constructed of lossless materials. The transmission can be smaller than unity as the result of the absorption by biological materials located near the resonator structure or due to unwanted losses associated with poor confinement of light within the resonator (see Little & Chu, “Estimating surface-roughness loss and output coupling in microdisk resonators,” Opt. Lett. 21, 1390-1392(1996), which is hereby incorporated by reference in its entirety). Equations (1)-(3) can be solved simultaneously to find that the input and output fields are related by

$$\frac{{E}_{2}}{{E}_{1}} = \frac{i t τ exp ( ⅈ φ )}{1 - r τexp ( ⅈ φ )} \qquad Eq . ( 4 )$$

The absolute square of this quantity is the buildup factor B introduced above:

$$B = \frac{{I}_{2}}{{I}_{1}} = {\vert \frac{{E}_{2}}{{E}_{1}} \vert}^{2} = \frac{( 1 - {τ}^{2} ) {τ}^{2}}{1 - 2 r τ cos ( φ ) + {r}^{2} {τ}^{2}} \qquad Eq . ( 5 )$$

It should be noted that the buildup takes on its maximum value

$${B}_{max} = \frac{1 + τ}{1 - r} \qquad Eq . ( 6 )$$

under conditions such that the incident light is resonant with the structure (that is, φ=2πm for integral m) and attenuation is negligible (τ=1).

The transmission through the waveguide that is coupled to the disk resonator can also be determined. The amplitude transmission E₃/E₁ is resolved by combining Eqs. (1) and (4) to obtain

$$\frac{{E}_{3}}{{E}_{1}} = exp [ ⅈ ( π + σ ) ] \frac{τ - r exp ( - ⅈ φ )}{1 - r τexp ( ⅈ φ )} \qquad Eq . ( 7 )$$

The intensity transmission factor T is given by the squared modulus of this quantity or by

$$\frac{{I}_{3}}{{I}_{1}} = {\vert \frac{{E}_{3}}{{E}_{1}} \vert}^{2} = \frac{{τ}^{2} - 2 r τ cos φ + {r}^{2}}{1 - 2 r τcos φ + {r}^{2} {τ}^{2}} \qquad Eq . ( 8 )$$

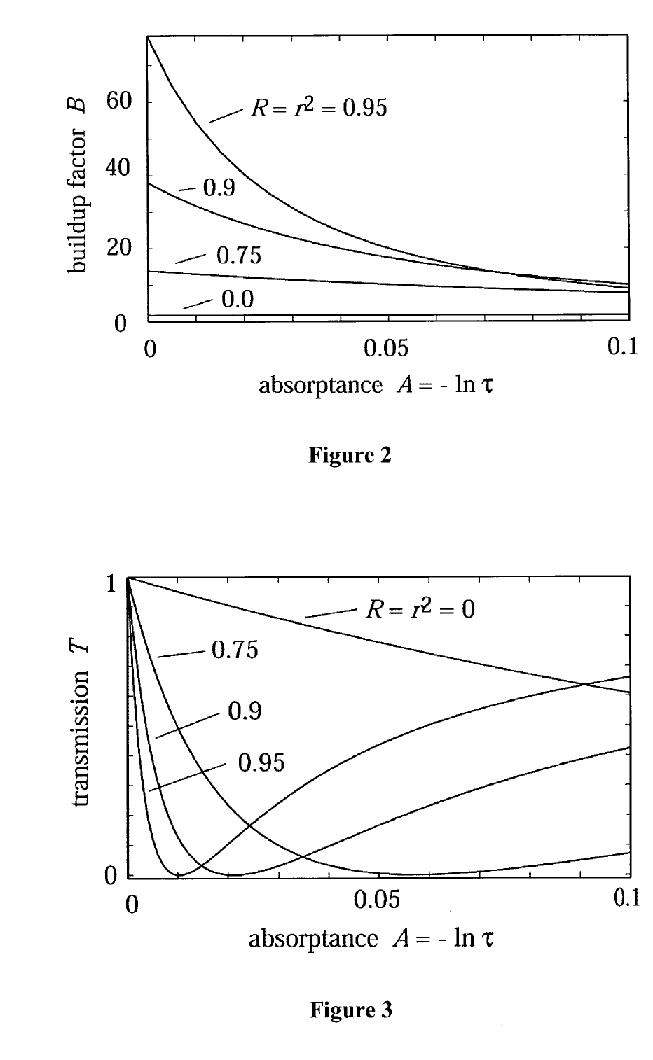

Some of the predictions of the model just presented are shown in FIGS. 2-4. FIG. 2 shows how the buildup factor B depends on the presence of absorption occurring within the resonator structure. The buildup factor is plotted against the single-pass absorption A, defined in terms of the single-pass transmission such that τ²=exp(−A), and results are shown for several values of the coupling coefficient R=r². Thus, for coupling coefficients R approaching unity, the buildup factor B decreases rapidly with increasing absorption. This variation of the internal intensity with the internal absorption A can be monitored in any of several different ways to form a biosensor. For example, the intensity of inevitable scattering of light out of the resonator can be measured to monitor the intensity of the circulating light. Alternatively, the intensity of the light leaving the output port of the device can be measured. This possibility is analyzed next.

FIG. 3 shows how the transmitted intensity varies with the presence of internal absorption within the disk resonator. More precisely, the resonator transmission T=|E₃/E₁|² is plotted against the single-pass absorption A=−ln τ for several values of the coupling coefficient R=r². Note that in all cases the device transmission initially decreases with increasing absorption and that for some particular value of the absorption the transmission drops to zero. Under these conditions, the resonator is said to be critically coupled to the optical waveguide. The vanishing of the transmission can be traced to a complete destructive interference of the two contributions (see Eq. (1)) to the transmitted field. Either of these properties can be used to construct a biosensor. For example, examination of Eq. (8) shows that the transmission drops to zero when the absorption has the value

A=−ln R Eq. (9)

In addition, it can be shown that the change in transmission with absorption is maximum for small absorption under resonance conditions and has the value

dT/dA=B Eq. (10)

where B is the buildup factor introduced above.

It is also possible to construct a biosensor based on the change in phase of the circulating optical power resulting from the change in refractive index of the disk when biological materials fall onto its surface. A device that can function in this manner is shown in FIG. 1B. Two output ports are required in this case because, in the absence of absorption, the transmission of the device is equal to unity for any value of the single-pass phase φ as a result of energy conservation. For the device of FIG. 1B, the input power is split between the two output ports in a manner that depends on the phase φ. A simple calculation analogous to that shown above indicates that the transmission functions for this device are given in the absence of absorption by

$$\begin{matrix}\begin{matrix}{T}_{5} = \frac{{I}_{5}}{{I}_{1}} \\ = \frac{{( 1 - {r}^{2} )}^{2}}{1 - 2 {r}^{2} cos φ + {r}^{4}} \\ = \frac{1}{1 + \frac{4 {r}^{2}}{{( 1 - {r}^{2} )}^{2}} {sin}^{2} \frac{1}{2} φ}\end{matrix} & Eq . ( 11 ) \\ {T}_{3} = \frac{{I}_{3}}{{I}_{1}} = 1 - {T}_{5} & Eq . ( 12 )\end{matrix}$$

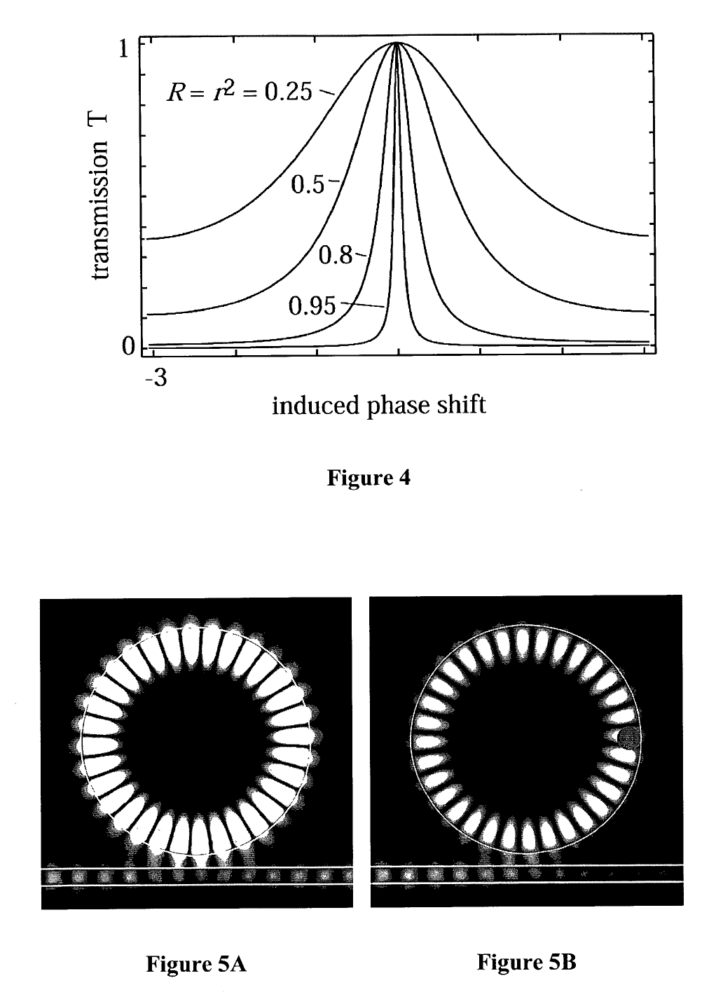

Equations (11) and (12) are a form of the Airy formula, which is encountered in the theory of the Fabry-Perot interferometer. Shown in FIG. 4 is a plot of the resonator transmission T=|E₃/E₁|² against the single-pass phase shift Δφ for several values of the coupling coefficient R=r². These curves were calculated under the assumption that the coupling coefficient was the same for the two waveguides. Thus, as the coupling coefficient R approaches unity, the central resonance become sharp. The extremely sensitive dependence of the transmission on the single-pass phase shift in this limit can be used to construct sensitive biosensors.

In a preferred embodiment, the surface of the disk resonator is functionalized with a layer containing one or more probes specific for a biological target. This layer can be applied to the active area of the disk resonator of FIGS. 1A-B to provide a specific binding of the type of material to be detected (Luff et al., “Integrated-optical directional coupler biosensor,” Opt. Lett. 21, 618-620 (1996); Luff et al., “Integrated optical Mach-Zehnder biosensor,” J. Lightwave Technol. 16, 583-592 (1998); Kolomenskii et al., “Sensitivity and detection limit of concentration and adsorption measurements by laser-induced surface-plasmon resonance,” Appl. Opt. 36, 6539-6547 (1997); Kolomenskii et al., “Surface-plasmon resonance spectrometry and characterization of absorbing liquids,” Appl. Opt. 39, 3314-3320 (2000); Rowe et al., “Array biosensor for simultaneous identification of bacterial, viral, and protein analytes,” Anal. Chem. 71, 3846-3852 (1999), each of which is hereby incorporated by reference in its entirety).

In estimating the minimum number of biological molecules that would have to fall onto the active area of the sensor to produce a reliable detection event, it assumed that the absorption cross sections of biological molecules are unlikely to change dramatically as a consequence of the weak (by chemical standards) binding associated, for example, with the binding of probes to their targets. Thus, the calculation presented here is likely to provide a reliable estimate of the sensitivity limits even in the more complicated situation in which a binding layer is employed. A representative value of the absorption cross section of a biological molecule is taken to be σ=2×10⁻¹⁶ cm², which is the measured value for the substance dopamine (see, e.g., Schulze et al., “Measurement of some small molecule and peptide neurotransmitters in-vitro using a fiber-optics probe with pulsed ultraviolet resonance Raman spectroscopy,” J. Neurosci. Methods 92, 15-24 (1999), which is hereby incorporated by reference in its entirety). It is expected that the absorption cross section for all single-electron, electric dipole transitions of molecules in the condensed phase would be of this order of magnitude. If A is the cross-sectional area of the mode of the optical field that is excited within the disk resonator, the fraction of the light that is absorbed per pass in interacting with this molecule is ƒ=ησ/A. Here η<1 is an efficiency factor that accounts for the fact that the molecule, located on the surface of the disk, does not experience the maximum intensity of the guided optical mode. It is expected that A would be of the order of (λ/n)², where n is the effective refractive index of the guiding region of the disk; and for η=0.5 and λ/n=1 μm, it is expected that f_{abs}=1×10⁻⁸. Such a small absorption is probably immeasurably small, but as noted above the total absorption is increased by the buildup factor B of the resonator, which could be as large as 10⁴. Because a change in transmission of 1% is believed to be measurable, this calculation suggests that as few as 100 biological molecules falling onto the biosensor will produce a detection event.

It is also useful to deduce some general formulas relating to the sensitivity of the biosensor of FIG. 1A. Equation (9) gives the condition under which the transmission of the sensor will drop to zero. This result can be expressed in terms of laboratory units as follows. Because A=−ln τ, where the single-pass transmission τ can be represented as τ=1−f_{abs}, for f_{abs}<<1 it is expected that A≈f_{abs}. Furthermore, because for R=r²≈1, the buildup factor B of Eq. (5) is given by B=2/(1−r), and therefore R≈1−4/B. Thus, the condition of Eq. (9) to produce a strict zero in the sensor transmission can be expressed as

$${f}_{a bs} = \frac{4}{B} \qquad Eq . ( 13 )$$

A more readily achieved condition is that the transmission through the device drops by a (smaller) prescribed amount. By means of a calculation analogous to that just presented, the amount of single-pass absorption required for the transmission to drop from unity to the value T (where it is assumed that 1−T<<1) is given by

$${f}_{a bs} = \frac{1 - T}{B} \qquad Eq . ( 14 )$$

For definiteness, the discussion of the detection sensitivity given above was cast in terms of the minimum number of individual molecules that can give rise to a detection event. It should be noted that many biological materials possess an absorption cross section much larger than that of simple molecules such as dopamine, which was used in this calculation. For example, a macromolecule such as a polypeptide chain could readily contain more than 100 side-chain units each with an absorption cross section comparable to that of dopamine, and thus should be detected. Larger entities such as viruses should also be detectable.

### Example 2

### Model Operation for Single Disk Resonator

The operation of the device of FIG. 1A was modeled by performing a numerical integration of Maxwell's equations for a region of space that includes the disk resonator and a portion of the waveguide. An example of the results of such a simulation is shown in FIGS. 5A-B, which compare the field distribution both in the absence and presence of an absorbing biological pathogen. For the particular values of the parameters used in this calculation, the presence of the pathogen leads to a dramatic change in the transmission characteristics of the device. In particular, in the presence of the pathogen the intensity of the circulating light field within the resonator decreases significantly and the transmitted intensity drops nearly to zero, illustrating the destructive interference mentioned in connection with FIG. 3.

The simulation shown in FIGS. 5A-B was calculated through use of the finite-difference time-domain method (Yee, “Numerical solution of initial boundary value problems involving Maxwell's equations in isotropic media,” IEEE Trans. Antennas Propag. 14, 302-307 (1966); Taflove & Hagness, Computational Electrodynamics, The Finite-Difference Time-Domain Method (Artech House, Boston, Mass., (2000); Hagness et al., “FDTD microcavity simulations: design and experimental realization of waveguide-coupled single-mode ring and whispering-gallery-mode disk resonators,” J. Lightwave Technol. 15, 2154-2165 (1997), each of which is hereby incorporated by reference in its entirety) and it entails solving Maxwell's equations numerically on a two-dimensional grid.

The following physical characteristics were assumed: a waveguide width of 0.4 μm, a gap between the guide and the disk of width 0.25 μm, a disk radius of 2.54 μm, a circular particle of radius 0.25 μm, and use of TM polarization (that is, E perpendicular to the plane containing the guide and disk). It is also assumed that the guide and disk have a dielectric constant of 4.0 and are separated by vacuum; these values mimic in two dimensions the three-dimensional nature of the waveguiding. The absorbing particle was modeled by assuming that the real part of its dielectric constant is also 4.0 and that its absorption can be described by a conductivity of 3000 Ω⁻¹ m⁻¹.

Based on these assumptions, the numerical simulation of these values lead to a buildup factor of 16 in the absence of the absorbing particle and a value of 3 in its presence. The results given in FIGS. 5A-B provide independent verification of the predictions provided above. The formalism underlying these predictions is based on the attribution of the phenomenological coefficients r and t to the coupling of light into and out of the disk resonator. The approach of this section is likely to prove extremely useful in the design of integrated photonic biosensors.

### Example 3

### Attaching Silane Coupling Agent to Silica Coated GaAs Substrate and Attaching Antibody Probe Thereto

Silica coated GaAs was first cleaned for 1 hour in 4M NaOH with stirring, followed by rinsing in deionized water and methanol and drying with nitrogen and thereafter for 1 hour at about 100° C. The cleaned material was then subjected to plasma cleaning for 15 minutes at 200 W, 1 torr O₂.

Aqueous alcohol deposition of trialkoxysilanes was conducted using an aqueous alcohol solution of 95 mL ethanol and 5 mL water, to which 2 mL of a trialkoxysilane was added. The solution was allowed to equilibrate for ˜5 minutes and then applied to the silica coated GaAs. The silica coated GaAs was then rinsed serially with ethanol, deionized water, and ethanol, and subsequently dried with nitrogen and then in an over for 30 minutes at about 100° C.

Once the primed surface has been prepared, a goat anti-O157 antibody will be attached thereto for use in recognizing pathogenic strains of E. coli O157. First, the silica coated GaAs will be incubated in 0.5 M Tris-(carboxyethyl) phosphine hydrochloride in 50 mM Tris-Cl pH 8.5 for 3.5 hours at 50° C. Then the silica coated GaAs will be washed in a coupling buffer (0.1 M Tris-Cl pH 7.5, 0.1 M NaCl, 1 mM EDTA) for 5 minutes at room temperature and rinsed with deionized water.

100 μl of antibody solution will be introduced into coupling buffer and incubated for 1 hour at room temperature. Primed silica coated GaAs will then be washed in a blocking buffer (1×phosphate buffered saline, 3% non-fat milk, 0.1% Triton X-100) and then washed again in the coupling buffer before being submerged in the antibody solution. The submerged primed GaAs will be incubated overnight at room temperature on a rocker. Finally, the now antibody-coated GaAs will be washed in phosphate buffered saline to remove unbound antibody.

After preparing the antibody-coated GaAs, the material will be exposed to E. coli under conditions allowing the goat anti-O157 antibody to bind the E. coli. Detection of the extent of E. coli binding will then be detected using conventional optical detectors.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A biosensor system for monitoring for a presence of a biological target, the system comprising:
a first optical waveguide optically coupled to a light source;
a first ring or disk resonator optically coupled to the first optical waveguide; and
a monitoring system that signals the presence of a biological target based on a detected change in one or more transfer characteristics of the first ring or disk resonator.

2. The biosensor system of claim 1 wherein the first ring or disk resonator is formed of a glass or crystalline semiconductor material.

3. The biosensor system of claim 1 wherein the resonator is a ring resonator.

4. The biosensor system of claim 1 wherein the resonator is a disk resonator.

5. The biosensor system of claim 1 further comprising one or more probes coupled to a surface of the first ring or disk resonator, the one or more probes binding to a target molecule.

6. The biosensor system of claim 5 wherein the one or more probes each bind the same target molecule.

7. The biosensor system of claim 5 wherein the one or more probes comprise first probes that bind a first target molecule and second probes that bind a second target molecule.

8. The biosensor system of claim 5 wherein the one or more probes is a non-polymeric small molecule.

9. The biosensor system of claim 5 wherein the one or more probes is a protein or polypeptide.

10. The biosensor system of claim 5 wherein the one or more probes is a nucleic acid molecule.

11. The biosensor system of claim 5 further comprising:
one or more coupling agents each comprising a first moiety attached to the surface of the first ring or disk resonator and a second moiety attached to one of the probes.

12. The biological biosensor system of claim 11 wherein the one or more coupling agents are silanes.

13. The biosensor system of claim 1 wherein the first ring or disk resonator comprises a first port.

14. The biosensor system of claim 13 wherein the first optical waveguide is a positioned between about 0.05 μm to about 0.5 μm from the first port.

15. The biosensor system of claim 13 wherein the first ring or disk resonator comprises a second port.

16. The biosensor system of claim 15 further comprising:
a second optical waveguide optically coupled to the first ring or disk resonator at the second port.

17. The biosensor system of claim 16 wherein the second optical waveguide is a positioned between about 0.05 μm to about 0.5 μm from the second port.

18. The biosensor system of claim 16 wherein the one or more transfer characteristics detected by the monitoring system is the effective refractive index of the ring or disk resonator or the optical losses associated with absorption or scattering out of the resonator.

19. The biosensor system of claim 1 wherein the monitoring system comprises an optical detector.

20. The biosensor system of claim 1 wherein the one or more transfer characteristics detected by the monitoring system is the effective refractive index of the ring or disk resonator, the optical losses associated with absorption or scattering out of the resonator, intensity of light leaving an output port, the phase change of the transmitted optical power, or a combination thereof.

21. The biological system of claim 1 further comprising:
a second ring or disk resonator optically coupled to the first optical waveguide,
wherein the monitoring system signals the presence of a biological material based on a detected change in one or more transfer characteristics of the first ring or disk resonator, the second ring or disk resonator, or a combination thereof.

22. A method of detecting the presence of a biological target in a sample, the method comprising:
exposing a biosensor system of claim 1 to a sample and
monitoring the first ring or disk resonator for a change in one or more transfer characteristics, wherein a change in the one or more transfer characteristics indicates adsorption of a biological target to a surface of the first ring or disk resonator and presence of the biological target in the sample.

23. The method of claim 22 wherein the sample is selected from the group consisting of blood, water, a suspension of solids in an aqueous solution, a cell suspension from a clinical isolate, and a tissue homogenate.

24. The method according to claim 22 wherein the transfer characteristic is the effective refractive index of the ring or disk resonator, the optical losses associated with absorption or scattering out of the resonator, the intensity of light leaving an output port, the phase change of the transmitted optical power, or a combination thereof.

25. A method of making the biosensor system of claim 5 comprising:
optically coupling a ring or disk resonator to one or more optical waveguides that are coupled to a light source and a monitoring system, and
attaching one or more probes to a surface of the ring or disk resonator.
